(19) United States
(12) Patent Application Publication
(10) Pub. No.: US 2026/0026710 A1
(43) Pub. Date: Jan. 29, 2026

(54) WEARABLE DEVICE WITH USER MOVEMENT ANALYSIS AND FALL RISK PREDICTION AND FALL DETECTION CAPABILITY

(71) Applicant: ULTRAHUMAN HEALTHCARE PVT LTD, Bangalore South Karnataka (IN)

(72) Inventors: Jaskaran SINGH, Patiala Punjab (IN); Mohit KUMAR, Layout Bangalore (IN); Vatsal SINGHAL, Bengaluru (IN); Adhit SHET, Bangalore (IN); Bhuvan SRINIVASAN, Bangalore (IN)

(21) Appl. No.: 19/348,219

(22) Filed: Oct. 2, 2025

Related U.S. Application Data
(63) Continuation of application No. PCT/IN2025/050365, filed on Mar. 13, 2025.

(30) Foreign Application Priority Data
Mar. 15, 2024 (IN) 202441019139

(51) Int. Cl. A61B 5/11; A61B 5/00

(52) U.S. Cl. CPC A61B 5/1117; A61B 5/6826; A61B 5/7267; A61B 5/7275; A61B 5/747; A61B 2503/08; A61B 2562/0219

(57) ABSTRACT

The present invention relates to a method and wearable device for monitoring and analyzing user movements to predict fall risk. The method involves retrieving motion data recorded by an Inertial Measurement Unit (IMU) and a pedometer, followed by processing the data once it exceeds a predefined threshold. A first trained machine learning (ML) model predicts the fall risk based on the processed data. A confidence score is determined from the fall prediction, and a second trained ML model analyzes successive data frames to confirm the fall if the score is above a threshold. If a fall is confirmed, a distress signal is transmitted. The wearable device comprises a processor and memory that stores program instructions to perform the method, including motion data retrieval, processing, fall prediction, confidence scoring, and fall confirmation, along with distress signal transmission. This invention enables timely fall detection and emergency response, improving user safety.

### FIELD OF INVENTION

The present invention relates to a wearable device, and more specifically, to a method and wearable device for monitoring and analyzing user movements for predicting and detecting fall of the user.

### BACKGROUND OF INVENTION

Falls are one of the leading causes of injury and death, particularly among elderly individuals and those with mobility impairments. According to studies, falls result in a significant number of hospitalizations, long-term disabilities, and even fatalities in this demographic. Elderly individuals, in particular, are more vulnerable to falls due to factors such as decreased muscle strength, poor balance, slowed reflexes, and vision impairments. Furthermore, individuals with chronic conditions such as arthritis, Parkinson's disease, or other neurological disorders face an elevated risk of falls. These falls can lead to serious injuries, including fractures, head trauma, and a decrease in overall quality of life.

In many cases, individuals who are at risk of falling live independently or in environments where immediate help may not be readily available. This makes it even more critical to identify and respond to fall risks in real-time, as the delay between a fall occurring and receiving assistance can lead to worsening injuries or even death. Despite the increasing recognition of this problem, conventional fall detection systems often fail to meet the required needs of users, both in terms of reliability and practicality.

One significant limitation of current fall detection solutions is the form factor of the devices. Traditional solutions for fall detection, such as pendants, belts, or wrist-worn devices, tend to be bulky and uncomfortable for long-term wear. Their size and appearance often deter individuals from consistently wearing them, particularly older adults who may feel self-conscious or constrained by these devices. As a result, these systems do not provide continuous, real-time monitoring, and fall risks may go undetected, leaving users vulnerable to injury. Moreover, these devices are often designed for specific settings or occasions (e.g., worn only during sleep or physical activity) and fail to offer the comprehensive, around-the-clock monitoring needed to predict falls before they occur.

Furthermore, many existing systems rely on simplistic algorithms or basic motion detection methods, such as detecting a sudden change in position or a fall-like motion. While these solutions may detect some types of falls, they often lack the sensitivity and precision required to differentiate between a fall and other activities that may involve similar body movements. For example, movements like sitting down, kneeling, or bending over may trigger false positives, leading to unnecessary alerts. On the other hand, subtle early warning signs of a fall, such as unsteady walking or changes in gait, are often missed due to a lack of advanced analysis or proper sensor integration. This lack of accuracy significantly reduces the effectiveness of conventional fall detection systems, as users may not be alerted when they need help most.

The inability to predict fall risk further exacerbates the problem. Many fall-related injuries are preceded by gradual changes in the user's walking patterns or balance, but conventional systems typically do not have the capability to assess these changes in real time. Early signs of instability, such as altered gait, slower walking speed, reduced step length, or unbalanced movements, often go undetected until a fall actually occurs. Without the ability to anticipate these issues, users are left without preventive interventions that could mitigate the risk of falling.

Additionally, existing systems are often not designed to provide real-time confirmation and alerts following a fall. Once a fall happens, immediate medical intervention is crucial to prevent further complications. Conventional systems may not efficiently detect falls or may struggle to confirm that a fall has occurred, especially if the individual is unconscious, immobile, or in a position where movement detection is not possible. The inability to confirm a fall and trigger timely assistance can result in a delay that significantly impacts the outcome for the individual.

Moreover, there is a demand for a system that continuously monitors user movement patterns to provide early indications of potential fall risks. The invention described herein seeks to address these challenges by providing a compact, comfortable, and reliable wearable device capable of predicting and detecting falls in real-time, offering a significant improvement over existing solutions in terms of both accuracy and usability.

### OBJECTIVES OF THE INVENTION

A general objective of the present invention is to provide a wearable device that is compact, comfortable, and unobtrusive, enabling individuals to wear it throughout their daily activities without discomfort, stigma, or interference, thus ensuring continuous monitoring of their movements for fall detection and risk prediction.

Another objective of the invention is to provide a method capable of accurately predicting an individual's fall risk by analyzing real-time motion data obtained from a combination of an Inertial Measurement Unit (IMU) and a pedometer.

Yet another objective of the invention is to provide a wearable device that can easily communicate with existing healthcare infrastructures, enabling seamless integration into monitoring systems used by caregivers, medical professionals, and emergency response teams.

Still another objective of the invention is to provide a method for accurately detecting and confirming falls when they occur, utilizing advanced machine learning techniques and successive data frames to analyze post-fall motion patterns.

### SUMMARY OF INVENTION

The summary is provided to introduce aspects related to a method and a wearable device for monitoring and analysis of user movements for predicting fall risk, and the aspects are further described below in the detailed description. This summary is not intended to identify essential features of the claimed subject matter, nor is it intended for use in determining or limiting the scope of the claimed subject matter.

In one aspect, the present invention relates to a method of monitoring and analysis of user movements for predicting fall risk. The method comprises steps of retrieving motion data of the user, wherein the motion data is recorded by an Inertial Measurement Unit (IMU) and a pedometer and then processing the retrieved motion data after the retrieved motion data has exceeded a pre-defined threshold. Further, the method also includes predicting a fall risk of the user using a first trained machine learning (ML) model, wherein the processed motion data is provided as input for the implementation of the first trained ML model. Subsequently, the method includes determining a confidence score of fall of the user based on analysis of the predicted fall risk. Further, the method comprises processing successive data frames using a second trained ML model to confirm the fall of the user if the confidence score is above a pre-defined level, wherein the fall is confirmed by analyzing post-fall motion indicated by the successive data frames and then transmitting a distress signal if fall of the user is confirmed.

According to an embodiment of the present invention, the step of predicting the fall risk of the user further comprises: calculating a stride length of the user from data obtained from the IMU during a walking session, wherein the walking session is identified based on step counts above a threshold value; assessing balance of the user by analyzing variability in acceleration and angular velocity data obtained from the IMU; assessing walking symmetry by analyzing deviation of consistency of forward and backward movement of hands obtained from the IMU; calculating steadiness of the user's walking pattern based on variability of the stride length, balance, and walking symmetry of the user; and calculating the fall risk based on the user's walking symmetry and the steadiness of the user's walking pattern.

In yet another embodiment of the present invention, the data obtained from the IMU to calculate the stride length includes acceleration data, vertical acceleration component, horizontal acceleration component, vertical displacement, and horizontal displacement.

According to an embodiment of the present invention, the walking session is identified if step count of the user is above the threshold without significant pauses.

In yet another embodiment of the present invention, the first trained ML model is trained using a reduced data obtained from processing of labelled frame data of multiple users, wherein the first trained data model is a neural-network based classifier.

According to an embodiment of the present invention, the labelled frame data of each user comprises fall taken class of data frames and non-fall taken class of data frames.

In yet another embodiment of the present invention, the processing of the labelled frame data involves the extraction of frequency and time domain features of the labelled frame data to reduce data dimensionality.

According to an embodiment of the present invention, the IMU is used for recording 6-axis motion data comprising acceleration and angular velocity along three axes (x, y, z), and the pedometer is used for determining step count of the user.

In yet another embodiment of the present invention, the processing of retrieved motion data involves at least one of formatting, cleaning, or arranging the motion data in a suitable format.

According to an embodiment of the present invention, the distress signal is transmitted by a connected device.

In yet another embodiment of the present invention, the second trained ML model is pre-trained based on data belonging to the first trained ML model.

In another embodiment, the present invention relates to a wearable device for monitoring and analysis of user movements for predicting fall risk. The wearable device comprising a processor; and a memory coupled with the processor. The memory stores program instructions configured to retrieve motion data of the user, wherein the motion data is recorded by an IMU and a pedometer, process the retrieved motion data after the retrieved motion data has exceeded a pre-defined threshold, predict fall risk of the user using a first trained ML model, wherein the processed motion data is provided as input for the implementation of the first trained ML model, determine a confidence score of fall of the user based on analysis of the predicted fall risk, process successive data frames using a second trained ML model to confirm the fall of the user if the confidence score is above a pre-defined level, wherein the fall is confirmed by analyzing post-fall motion indicated by the successive data frames and transmit a distress signal if fall of the user is confirmed.

According to an embodiment of the present invention, the program instructions for predicting the fall risk of the user is further configured to calculate a stride length of the user from data obtained from the IMU during a walking session, wherein the walking session is identified based on step counts above a threshold value, assess balance of the user by analyzing variability in acceleration and angular velocity data obtained from the IMU, assess walking symmetry by analyzing deviation of consistency of forward and backward movement of hands obtained from the IMU, calculate steadiness of the user's walking pattern based on variability of the stride length, balance, and walking symmetry of the user, and calculate the fall risk based on the user's walking symmetry and steadiness of the user's walking pattern.

In yet another embodiment of the present invention, the data obtained from the IMU to calculate the stride length includes acceleration data, vertical acceleration component, horizontal acceleration component, vertical displacement, and horizontal displacement.

According to an embodiment of the present invention, the walking session is identified if step count of the user is above the threshold without significant pauses.

In yet another embodiment of the present invention, the first trained ML model is trained using a reduced data obtained from processing of labelled frame data of multiple users, wherein the first trained data model is a neural-network based classifier.

According to an embodiment of the present invention, the labelled frame data of each user comprises fall taken class of data frames and non-fall taken class of data frames.

In yet another embodiment of the present invention, the processing of the labelled frame data involves the extraction of frequency and time domain features of the labelled frame data to reduce data dimensionality.

According to an embodiment of the present invention, the IMU is used for recording 6-axis motion data comprising acceleration and angular velocity along three axes (x, y, z), and the pedometer is used for determining step count of the user.

In yet another embodiment of the present invention, the processing of retrieved motion data involves at least one of formatting, cleaning, or arranging the motion data in a suitable format.

According to an embodiment of the present invention, the distress signal is transmitted by a device connected to the wearable device, and includes fall and non-fall taken frames.

In yet another embodiment of the present invention, the second trained ML model is pre-trained based on data belonging to the first trained ML model.

According to an embodiment of the present invention, the wearable device is a smart ring.

Other aspects and advantages of the invention will become apparent from the following description, taken in conjunction with the accompanying drawings, illustrating by way of example, the principles of the invention.

### BRIEF DESCRIPTION OF THE DRAWINGS

The accompanying drawings constitute a part of the description and are used to provide further understanding of the present invention. Such accompanying drawings illustrate the embodiments of the present invention which are used to describe the principles of the present invention. The embodiments are illustrated by way of example and not by way of limitation in the figures of the accompanying drawings in which like references indicate similar elements. It should be noted that references to “an” or “one” embodiment in this invention are not necessarily to the same embodiment, and they mean at least one. In the drawings:

FIG. 1 illustrates a block diagram of a wearable device capable of monitoring user movements and predicting fall risk of a user and detecting user fall, in accordance with an embodiment of the present invention;

FIG. 2 illustrates a module-based implementation of proposed wearable device, in accordance with an embodiment of the present invention;

FIG. 3 illustrates a flowchart that outlines the steps involved in detecting and confirming a fall, in accordance with an embodiment of the present invention;

FIG. 4 illustrates a block diagram showing various modules of a server connected to the wearable device, in accordance with an embodiment of the present invention.

### DETAILED DESCRIPTION OF THE INVENTION

The detailed description set forth below in connection with the appended drawings is intended as a description of various embodiments of the present invention and is not intended to represent the only embodiments in which the present invention may be practiced. Each embodiment described in this disclosure is provided merely as an example or illustration of the present invention, and should not necessarily be construed as preferred or advantageous over other embodiments. The detailed description includes specific details for the purpose of providing a thorough understanding of the present invention. However, it will be apparent to those skilled in the art that the present invention may be practiced without these specific details.

The proposed invention relates to a method and a wearable device capable of monitoring user movements and predicting fall risk and detecting user fall. The wearable device may be a smart watch, smart band, or an electronic ring. Although the details have been provided successively with reference to a smart ring merely for the sake of explanation, it must be understood that the invention could be fairly implemented in a similar manner using any other wearable device, such as the ones listed above.

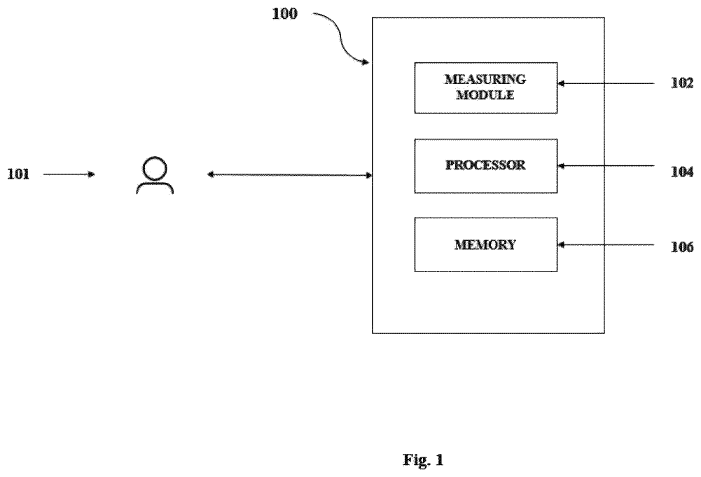

FIG. 1 illustrates a block diagram of a wearable device 100, such as a smart ring, capable of monitoring user movements, predicting fall risk of a user, in accordance with an embodiment of the present invention. The wearable device 100 may be made using a hypoallergenic material for allowing comfortable and continuous wear by a user 101. The device for monitoring and analysis of user movements for predicting fall risk 100 comprises a measuring module 102, a processor 104 and a memory 106 coupled with the processor. The measuring module 102 is used for capturing motion data of the user 101 of the device, and the real time processing unit 104 is used for monitoring and analyzing user movements for predicting fall risk.

The memory 106 stores program instructions configured to perform the method of monitoring and analysis of user movements for predicting fall risk. The method comprises steps of retrieving motion data of the user, wherein the motion data is recorded by the measuring module 106. The measuring module 106 includes at least one sensor, wherein the at least one sensor can be an Inertial Measurement Unit (IMU) and a pedometer. The method then includes step of processing the retrieved motion data after the retrieved motion data has exceeded a pre-defined threshold. Further, the method also includes predicting a fall risk of the user using a first trained machine learning (ML) model, wherein the processed motion data is provided as input for the implementation of the first trained ML model. Subsequently, the method includes determining a confidence score of fall of the user based on analysis of the predicted fall risk. Further, the method comprises processing successive data frames using a second trained ML model to confirm the fall of the user if the confidence score is above a pre-defined level, wherein the fall is confirmed by analyzing post-fall motion indicated by the successive data frames and then transmitting a distress signal if fall of the user is confirmed. The present invention provides a more advanced, reliable, and discreet solution that addresses the shortcomings of traditional fall detection and prevention technologies. The present invention not only accurately predicts fall risks based on real-time data but also offers high-precision detection once a fall occurs, minimizing false positives and negatives. Importantly, the present invention can be unobtrusive, allowing users to wear it consistently throughout their daily activities without discomfort or stigma.

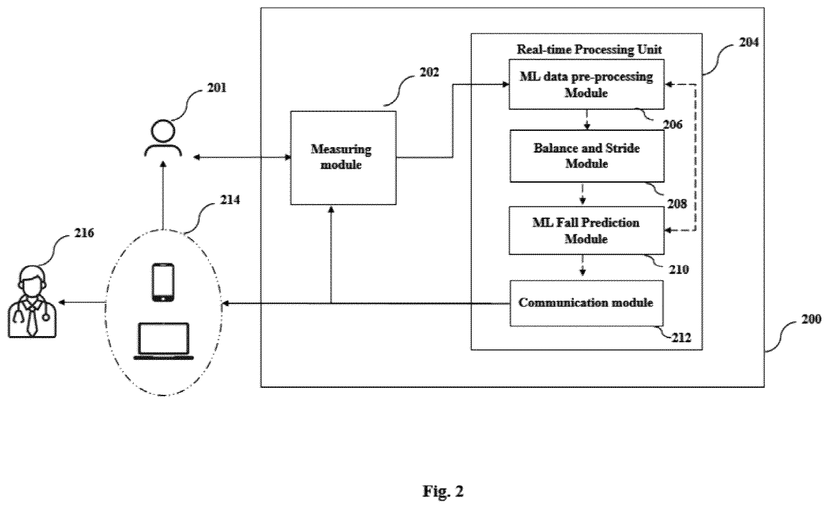

FIG. 2 illustrates a module-based implementation of proposed wearable device, in accordance with an embodiment of the present invention. The modules illustrated herein relate to the different functionalities of the wearable device, and the implementation of functionality of each module is achieved based on performing of the corresponding instructions stored in the memory of the wearable device 200. The instructions are performed by the processor of the wearable device 200. The modules associated with the different functionality of the wearable device 200 include a measuring module 202 and a real time processing unit 204, where the real time processing unit 204 is used for monitoring user movements and determining several details such as user stride, balance, and fall risk.

The measuring module 202 comprises at least one sensor for acquiring motion data of the user 201. In other embodiments, the at least one sensor may be an IMU or a pedometer, or both. The data acquired by the IMU of the measuring module 202. The IMU is used for recording 6-axis motion data comprising acceleration and angular velocity along three axes (x, y, z), and the pedometer is used for determining step count of the user.

The data acquired by the measuring module 202 is retrieved the real-time processing unit 204. The real-time processing unit 204 includes a ML data pre-processing module 206, a balance and stride module 208, a ML fall prediction module 210 and a communication module 212. The real-time processing unit 204 is used for monitoring and analysis of user movements based on the input retrieved from the measuring module 202 for predicting fall risk. The real-time processing unit 204 retrieves motion data of the user, wherein the motion data is recorded by the measuring module 202 having the IMU and the pedometer. The processing of retrieved motion data involves at least one of formatting, cleaning, or arranging the motion data in a suitable format.

The ML data pre-processing module 206 processes the retrieved motion data after the retrieved motion data has exceeded a pre-defined threshold. The pre-defined threshold is a critical component in ensuring the accurate detection of genuine walking motions while filtering out random or non-walking movements. This threshold is set to maintain system accuracy by avoiding false positives and ensuring reliable walking pattern detection. In the system's walking detection criteria, the primary requirement is the completion of at least 20 consecutive steps without significant pauses exceeding 2 seconds, as specified in the patent. Additionally, the walking cadence is restricted to a range of 70-150 steps per minute, aligning with the normal human walking pace. This range is essential as slower cadences may indicate potential instability, while faster cadences could suggest running or jogging activities, which are to be excluded from walking detection. Motion data collection is triggered once the pedometer detects a minimum of 20 steps taken at a consistent cadence within the prescribed walking range, with no long pauses detected between steps, further ensuring that only genuine walking activity is captured for analysis.

Successively, the ML data pre-processing module 206 may provide the motion data to the ML fall prediction module 210. The ML fall prediction module 210 includes a trained ML model and a prediction management model. The ML fall prediction module 210 may load the trained ML model and the prediction management model onto a ML co-processor. The trained ML model may be a neural network classifier capable of predicting a fall risk, i.e., a probability of fall of the user by processing the motion data. Accordingly, the ML fall prediction module 210 predicts a fall risk of the user using a first trained ML model, wherein the processed motion data is provided as input for the implementation of the first trained ML model. The first trained ML model is trained using a reduced data obtained from processing of labelled frame data of multiple users, wherein the first trained data model is a neural-network based classifier. The labelled frame data of each user comprises fall taken class of data frames and non-fall taken class of data frames for training the first trained ML model. Further, the processing of the labelled frame data involves the extraction of frequency and time domain features of the labelled frame data to reduce data dimensionality.

A prediction result, i.e., the fall risk, determined by the trained ML model may be analysed using the prediction model for determining a course of action to be taken. In case the prediction result does not indicate falling of the user, no action is taken. Alternatively, if the prediction result indicates falling of the user with a sufficient confidence score, successive data frames are processed as post-fall taken frames by a second trained ML model. The second trained ML model ascertains whether the user has truly taken a fall by analysing their post-fall motion behaviour, as the user is likely to show certain characteristic patterns such as not being able to stand up or not walking, etc.

A confidence score of fall of the user is determined based on analysis of the predicted fall risk. The confidence score is calculated using a two-step ML-based classification process to determine whether the user has experienced a fall. In the initial classification, data from successive frames is processed through the ML model that outputs a score between 0 and 1, indicating the likelihood of a fall. A threshold of 0.5 is applied to classify events as either “fall taken” or “normal.” If the initial model predicts a “fall taken,” then post-fall confirmation is initiated, where motion data is further analyzed by a second model. The ML fall prediction module 210 processes successive data frames using the second trained ML model to confirm the fall of the user if the confidence score is above a pre-defined level, wherein the fall is confirmed by analyzing post-fall motion indicated by the successive data frames. The primary training process for the second trained ML model should align with that of the first trained model. The second trained ML model is trained on data frames with the following classes: non-post-fall-taken and post-fall-taken. This second trained ML model assesses the likelihood that the user is in a post-fall state, considering factors such as limited motion and distinct movement patterns. Again, a 0.5 threshold is applied to classify the result as either “fall confirmed” or “no fall taken.” A fall is confirmed only if both models classify the event as a fall, providing a reliable and accurate determination. Both models employ binary classification, using a threshold of 0.5 to distinguish between fall-related events and normal activities.

Upon finding a sufficient confidence score related to falling of the user, the prediction module 210 communicates with the communication module 212 to transmit a distress signal. The communication module 212 may send the distress signal to a user device 214, such as a smartphone, tablet, laptop, etc. The distress signal may include the fall and non-fall taken frames. In certain embodiments, the user device 214 may be used by a second user 216 such as a physician, doctor, or medical care-giver for keeping track of the user movements of the user 201.

The measuring module 202 also includes the pedometer that may detect that the user 201 is walking, upon identifying an increase in number of steps taken by the user. Upon such detection, motion frame data is passed to the balance and stride module 208 for checking symmetries and patterns in forward and backward hand movements of user during the walk. Such data of the forward and the backward hand movements is used to determine the user's balance, stride length, and a fall risk. Specifically, the data obtained from the IMU to calculate the stride length includes acceleration data, vertical acceleration component, horizontal acceleration component, vertical displacement, and horizontal displacement. The user's balance, stride length, and the fall risk are determined at regular intervals, stored in the memory 106, and sent to the connected user device 214 whenever needed.

The balance and stride module 208 calculates a stride length of the user 201 from data obtained from the IMU during a walking session, wherein the walking session is identified based on step counts above a threshold value. The balance and stride module also assess balance of the user by analyzing variability in acceleration and angular velocity data obtained from the IMU. Balance is a critical factor in assessing movement stability, and its measurement plays an important role in identifying walking irregularities. In the balance and stride module, balance is determined by calculating the variation in arm movements, which are monitored using accelerometer readings. These readings capture three axes of movement: ax for side-to-side arm swing, ay for forward-backward swing, and az for up-down motion. For each movement window, the deviation from the typical arm swing pattern is calculated, with motion separated based on its directionality. The resulting data is used to compute a Walking Balance Score, which reflects the level of stability in a user's walking pattern. A higher balance score indicates a more stable walking pattern, while a lower score suggests potential instability, offering valuable insights into the user's gait and movement stability.

The balance and stride module 208 also assess walking symmetry by analyzing deviation of consistency of forward and backward movement of hands obtained from the IMU. The walking session is identified if step count of the user is above the threshold without significant pauses. Walking symmetry is a key factor in evaluating the consistency of a user's gait, and it is assessed using data the IMU that tracks arm motion. The basic movement data is derived from accelerometer readings along three axes: ax for lateral (side) acceleration, ay for forward-backward acceleration, and az for vertical acceleration. Swing cycle analysis is conducted by identifying the forward swing, defined by the peak positive ay during the swing, and the backward swing, represented by the peak negative ay. The cycle time is calculated as the time between consecutive forward swing peaks. Swing symmetry is determined by analyzing each complete arm swing cycle, where a score near 1.0 indicates a balanced forward and backward swing, while lower scores suggest asymmetrical movement. The overall symmetry score is derived by averaging the symmetry scores across multiple cycles. A lower symmetry score indicates an imbalance in the user's walking pattern, which may point to gait irregularities or instability.

The ML fall prediction module 210 calculates steadiness of the user's walking pattern based on variability of the stride length, balance, and walking symmetry of the user. Walking steadiness is a comprehensive metric that evaluates balance, symmetry, and stride variability to detect irregularities in a user's walking pattern. The input components used to calculate walking steadiness include the Balance Score (B), which reflects arm movement control during walking (ranging from 0 to 1), the Symmetry Score (S), which measures the evenness of arm swings (ranging from 0 to 1), and Stride Length Variability (V), which is determined by calculating the variability in stride lengths based on the periods of arm swings. The Steadiness Score is then computed by combining these components, with a high score indicating stable and controlled movement, while a low score suggests potential instability or irregularities in the user's gait. This metric provides valuable insights into the user's overall walking stability and can help identify any deviations from normal walking patterns.

Fall risk assessment involves evaluating multiple factors that contribute to a user's movement stability. This assessment is crucial for predicting the likelihood of a fall. The components used in calculating the Fall Risk Score include the Balance Score (B), which measures the control of movement (ranging from 0 to 1), the Symmetry Score (S), which assesses the evenness of movement (ranging from 0 to 1), the Steadiness Score (T), representing the overall stability of the walking pattern (ranging from 0 to 1), and the Stride Length Variability (V), which evaluates the consistency of steps (ranging from 0 to 1). Additionally, Movement Speed (M), derived from arm swing frequency (ranging from 0 to 1), and Age (A) are taken into account. Age is categorized as follows: under 40 years (score=1), 40-55 years (score=0.7), 55-65 years (score=0.4), and 65+ years (score=0). The Fall Risk Score is calculated by combining these factors, with higher scores indicating a greater risk of falling. This method ensures an objective assessment of fall risk by incorporating both measurable movement patterns and relevant physiological factors.

In another embodiment of the present invention, the wearable device may include an Inertial Measurement Unit, Bluetooth Low Energy (BLE) communication unit, pedometer, Central Processing Unit (CPU), and a battery and charging unit/circuitry. The IMU unit may include a gyroscope and an accelerometer. Further, the CPU may exist and operate alongside a Machine Learning (ML) co-processor. By leveraging advanced sensing technologies, such as IMUs and pedometers, and incorporating machine learning algorithms, the wearable device of the present invention can accurately monitor gait and balance, detect deviations, and predict the likelihood of a fall before it happens. This proactive approach to fall prediction could enable caregivers or emergency responders to intervene early, reducing the chances of a serious injury or fatality.

In addition to predicting fall risk, a system of the present invention can be capable of confirming a fall with high confidence once it occurs, even if the individual is unable to communicate. By processing successive data frames and analyzing post-fall motion, such a system can accurately identify a fall and immediately transmit distress signals to caregivers, medical personnel, or emergency services

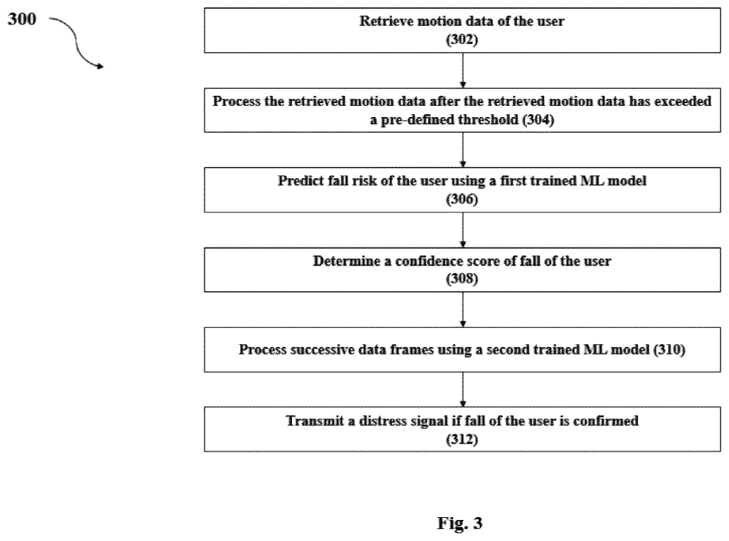

FIG. 3 illustrates a flowchart that outlines the steps involved in detecting and confirming a fall, in accordance with an embodiment of the present invention. The method 300 begins with step 302 of retrieving the motion data. This data is captured by the IMU, which records 6-axis motion data (acceleration and angular velocity along three axes: X, Y, and Z), and the pedometer, which provides step count data. The motion data may include acceleration, angular velocity, step count, and other relevant metrics.

In the method, step 304 involves processing of the retrieved motion data when the retrieved motion data exceeds a predefined threshold. The processing can include tasks such as data formatting, cleaning, and arranging the data in a suitable format to be fed into the trained ML models.

At step 306, the fall risk of the user is predicted based on processing of the data by a trained ML model. A first trained ML model is used to predict the fall risk based on the processed motion data. This model may be a neural network-based classifier that has been trained using labeled data from multiple users, where the labelled data includes fall and non-fall.

In the method, step 308 involves calculating a confidence score of a potential fall based on the predictions of the first trained ML model. The confidence score indicates the likelihood that a fall has occurred or is imminent. This confidence score is a threshold value used for the next step of confirmation.

In step 310, a second trained ML model processes successive data frames to confirm the fall if the confidence score exceeds a predefined level. This confirmation process involves analyzing post-fall motion data from the successive data frames, such as detecting the characteristic movements of a fall and assessing the body's position after a fall.

Step 312 involves transmitting a distress signal to a designated device, such as a mobile phone or emergency contact, once the fall has been confirmed. The distress signal contains fall-related information, including both fall and non-fall data frames, which may be used to assess the situation in greater detail.

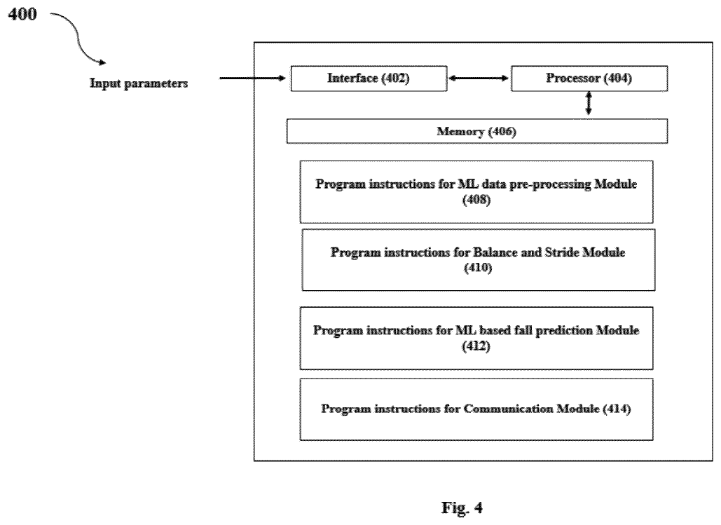

In one embodiment, the prediction and detection of fall of a user may be performed by a server connected to the wearable device. In such an embodiment, the motion data may be collected by the wearable device using its sensors, and the data may be provided to the server. FIG. 4 illustrates a block diagram showing various modules of the server connected to the wearable device, in accordance with an embodiment of the present invention. The motion data of the wearable device may be retrieved by the server 400, connected to the device. The server 400 may comprise an interface 402, a processor 404, and a memory 406. The motion data of the user can be measured from a plurality of measuring modules, such as IMU and pedometer, and the same is referred as input parameters in FIG. 4. The input parameters may be provided to the server 400 through the interface 402. The memory 406 may store program instructions for ML data pre-processing module 408, balance and stride module 410, ML based fall prediction module 412, and communication module 414 in order to monitor user movements, predict fall risk and detect user fall.

A method performed by the server 400 of monitoring user movements and determining several details such as user stride, balance, and fall risk is explained successively. The IMU may be used for continuously recording 6-axis motion data and providing the motion data to the processor 404. The motion data may also be stored in a memory 406 of the server 400. When an amount of the motion data exceeds a predefined threshold, the processing of the motion data is performed by the server based on program instructions for the ML data pre-processing module 408. Specifically, the program instructions for ML data pre-processing module 408 may format, clean, and arrange the motion data into a suitable format useable for further processing. Successively, the motion data is provided to the server for predicting fall risk based on program instructions for ML fall prediction module 412. The program instructions for ML fall prediction module may load a trained ML model onto the ML co-processor coupled to the processor 404 of the server 400. The trained ML model may be a neural network classifier capable of predicting a fall risk, i.e., a probability of fall of the user by processing the motion data. A prediction result, i.e., the fall risk, determined by the trained ML model may be analysed using the prediction model for determining a course of action to be taken. In case the prediction result does not indicate falling of the user, no action is taken.

The pedometer of the wearable device may detect that the user is walking, upon identifying an increase in number of steps taken by the user. Upon such detection, motion frame data is passed to the server for checking symmetries and patterns in forward and backward hand movements of user during the walk based on program instructions on the balance and stride algorithm module 410. Upon finding a sufficient confidence score related to falling of the user, the server based on program instructions for the fall prediction module 412 communicates with the communication module to transmit a distress signal. The program instructions for communication module 414 is configured to send the distress signal to a user device 214, such as a smartphone, tablet, laptop, etc.

The user's fall risk is calculated using the asymmetry and steadiness metrics from the previous steps, with higher levels of asymmetry and lower levels of steadiness indicate an increased fall risk.

The present invention offers several key advantages that enhance user safety and system reliability. It enables real-time monitoring and analysis of user movements, facilitating continuous tracking of the user's gait and balance for prompt intervention. The invention also provides accurate prediction of fall risk by evaluating multiple motion parameters, allowing for early detection of potential instability in the user's walking pattern. In the event of a fall, timely fall confirmation and distress signal transmission ensure that immediate action can be taken, improving the user's safety and response time. Moreover, the system is designed for seamless integration into wearable devices, offering an easy-to-use, comfortable solution that does not interfere with the user's daily activities or mobility. The use of machine learning models further enhances the system's accuracy over time, particularly in confirming falls, which significantly increases the overall reliability and effectiveness of the fall detection system.

We claim:

1. A wearable device for monitoring and analysis of user movements for predicting fall risk, the wearable device comprising:
a processor; and
a memory coupled with the processor, wherein the memory stores program instructions configured to:
retrieve motion data of the user, wherein the motion data is recorded by an IMU (Inertial Measurement Unit) and a pedometer;
process the retrieved motion data after the retrieved motion data has exceeded a pre-defined threshold;
predict fall risk of the user using a first trained ML model, wherein the processed motion data is provided as input for the implementation of the first trained ML model;
determine a confidence score of fall of the user based on analysis of the predicted fall risk;
process successive data frames using a second trained ML model to confirm the fall of the user if the confidence score is above a pre-defined level, wherein the fall is confirmed by analyzing post-fall motion indicated by the successive data frames; and
transmit a distress signal if fall of the user is confirmed.

2. The wearable device as claimed in claim 1, wherein the program instructions for predicting the fall risk of the user is further configured to:
calculate a stride length of the user from data obtained from the IMU during a walking session, wherein the walking session is identified based on step counts above a threshold value;
assess balance of the user by analyzing variability in acceleration and angular velocity data obtained from the IMU;
assess walking symmetry by analyzing deviation of consistency of forward and backward movement of hands obtained from the IMU;
calculate steadiness of the user's walking pattern based on variability of the stride length, balance, and walking symmetry of the user; and
calculate the fall risk based on the user's walking symmetry and steadiness of the user's walking pattern.

3. The wearable device as claimed in claim 2, wherein the data obtained from the IMU to calculate the stride length includes acceleration data, vertical acceleration component, horizontal acceleration component, vertical displacement, and horizontal displacement.

4. The wearable device as claimed in claim 2, wherein the walking session is identified if step count of the user is above the threshold without significant pauses.

5. The wearable device as claimed in claim 1, wherein the first trained ML model is trained using a reduced data obtained from processing of labelled frame data of multiple users, wherein the first trained data model is a neural-network based classifier.

6. The wearable device as claimed in claim 5, wherein the labelled frame data of each user comprises fall taken class of data frames and non-fall taken class of data frames.

7. The wearable device as claimed in claim 5, wherein the processing of the labelled frame data involves the extraction of frequency and time domain features of the labelled frame data to reduce data dimensionality.

8. The wearable device as claimed in claim 1, wherein the IMU is used for recording 6-axis motion data comprising acceleration and angular velocity along three axes (x, y, z), and the pedometer is used for determining step count of the user.

9. The wearable device as claimed in claim 1, wherein the processing of retrieved motion data involves at least one of formatting, cleaning, or arranging the motion data in a suitable format.

10. The wearable device as claimed in claim 1, wherein the distress signal is transmitted by a device connected to the wearable device, and includes fall and non-fall taken frames.

11. The wearable device as claimed in claim 1, wherein the second trained ML model is pre-trained based on data belonging specifically to the first trained ML model.

12. The wearable device as claimed in claim 1, wherein the wearable device is a smart ring.
